(19) United States
(12) Patent Application Publication
(10) Pub. No.: US 2026/0365971 A1
(43) Pub. Date: Nov. 19, 2026

(54) Method of producing a preparation of secretory microvesicles from stem cells and use of the preparation of the secretory microvesicles produced by this method

(71) Applicant: UNIWERSYTET MEDYCZNY W BIALYMSTOKU, Bialystok (PL)

(72) Inventors: Marlena TYNECKA, Bialystok (PL); Andrzej ELJASZEWICZ, Studzianki (PL); Marcin MONIUSZKO, Olmonty (PL)

(21) Appl. No.: 19/477,243

(22) PCT Filed: Apr. 19, 2024
(86) PCT No.: PCT/IB2024/053826

§ 371 Date: Oct. 21, 2025

(30) Foreign Application Priority Data
Apr. 21, 2023 (PL) P.444517

(51) Int. Cl. C12N 5/073; A61K 35/28; C12N 13/00

(52) U.S. Cl. CPC C12N 5/0605; A61K 35/28; C12N 13/00; C12N 2501/999

(57) ABSTRACT

The present invention relates to a method of obtaining a biological preparation comprised of secretory microvesicles (MV) of mesenchymal stem cells (MSCs) and to the use of the preparation of secretory microvesicles, as obtained by the present method, for the treatment of respiratory tract diseases, including severe asthma.

The present invention relates to a method of obtaining a biological preparation comprised of secretory microvesicles (MVs) of mesenchymal stem cells (MSCs) in order to utilize them for the treatment of respiratory tract diseases, including asthma. In a further aspect, the present invention also relates to the preparation of secretory microvesicles, as obtained by the present method.

In accordance with a report by the World Health Organization, there are currently more than 300 million people suffering from asthma worldwide. Moreover, the lack of an effective control of inflammation in the course of asthma can lead to exacerbations of the disease, causing about 250 thousand deaths every year. Patients who do not respond to the standard treatment based on the use of glucocorticosteroids consume about half of all the funds intended for the treatment of this disease.

In the United States, the expenses related to the treatment of asthma amount to even up to 80 billion dollars every year. Moreover, an analysis of the costs related to the medical care for patients with uncontrolled asthma over the consecutive 20 years has shown a budget burden in the amount of 300.6 billion dollars or $963.5 billion dollars, taking into account the indirect costs. In Poland, about 4 million people suffer from asthma, with the cost of medical care per patient being estimated to be about 17.5 thousand zlotys. The amount of the expenses related to medical care for asthmatics increases with the stage and severity of the disease. The biggest clinical challenge is posed by the patients who do not respond to the available forms of therapy. Therefore, a substantial need exists to implement new solutions which will enable the development of new therapies. Various endotypes are distinguished in the pathogenesis of asthma. Lower respiratory tract inflammation with a predominance of neutrophilic infiltration (neutrophilic asthma, not directly dependent on the Th2 cell activity, defined as T2-independent), eosinophilic asthma (Th2-dependent, T2-dependent inflammation), and a mixed eosinophilic-neutrophilic endotype. The therapeutic options currently available for patients with neutrophilic asthma and for some patients with the mixed endotype are clearly limited compared to the strategies proposed for eosinophilic asthma. Inhaled corticosteroids (ICS) constitute the gold therapeutic standard enabling respiratory tract inflammation to be effectively controlled in majority of the patients, however, about 10-15% of the affected patients do not respond to the treatment used. Moreover, the introduction of targeted biological therapies directly modulating the immune response by blocking the activity of Th2 inflammation-associated cytokines, namely IL-4, IL-5 and IL-13, allowed for a relatively good control of the symptoms and exacerbations occurring in the course of eosinophilic asthma. However, an increased proportion of neutrophils occurring in about 50% of the patients is responsible for the severe course of the disease, as well as a higher incidence of steroid resistance. In connection with the above, it is necessary to develop new therapeutic strategies enabling the inflammation to be controlled in the patients with severe steroid-resistant asthma. It should be emphasized that chronic, suboptimally controlled respiratory tract inflammation can lead to the development of irreversible structural changes in the pulmonary tissue, referred to as remodeling and leading to a limitation of the respiratory function of the lungs.

The therapeutic potential as well as the immunosuppressive properties of cell transplants utilizing the MSCs have been repeatedly confirmed in preclinical models, and in the past years, the first clinical studies involving the utilization of these cells in asthma and other chronic inflammatory respiratory system diseases have been recorded. The studies to date assessing the therapeutic potential of the MSCs have been conducted on cells derived mainly from the bone marrow or umbilical cord blood. However, over the recent years, these cells have also been identified and isolated from nearly all the body tissues, including, among others, the adipose tissue, Wharton's jelly, the synovial fluid, the dental pulp as well as the adipose tissue. The laboratory procedures of isolation and culturing of the MSCs from different tissue sources are similar to each other and include mechanical comminution of the tissue, enzymatic degradation of collagen fibers, and culturing employing nutrient media (a medium) promoting their growth and proliferation. According to literature reports, the MSCs are phenotypically and functionally diverse. The individual subpopulations (subtypes) of the MSCs may differ slightly in immunosuppressive (anti-inflammatory) activity and regenerative potential. The heterogeneity of the MSC cells is one of the reasons preventing their wide therapeutic utilization. However, it should be noted that the anti-inflammatory activity of the MSCs is acquired under the influence of stimulation with pro-inflammatory factors.

A confirmation of the promising properties of the MSCs in pre-clinical models of chronic inflammatory diseases has allowed for the continuation of work on the assessment of the effectiveness of action and safety of use of the MSCs in clinical trials. Unfortunately, the finalization of the clinical studies has not led to the registration of a cell therapy utilizing MSCs. However, it should be emphasized that in an overwhelming majority, these solutions have relied on the concept of a direct activation of the MSCs in the inflammatory environment of the tissues of the recipient. The adoption of such an approach does not solve the problem of the heterogeneity of the MSCs attained from different sources and from different donors. Additionally, it does not take into account the heterogeneity of the environment of the lungs in the course of asthma. Currently, 3 main endotypes (inflammatory phenotypes of asthma) are distinguished: 1) an eosinophilic one, associated with the Th2 lymphocyte response (T2 asthma); 2) a neutrophilic one, characterized by the Th1/Th17 response (T2-independent asthma), and 3) a mixed one, combining the features of both inflammatory phenotypes. Considerable differences in the composition of the inflammatory environment of the lower respiratory tract between patients may be a cause of a heterogeneous and/or ineffective activation of the cells and, in connection with this, lower their therapeutic potential. Moreover, the extremely complex and dynamically changing microenvironment of the lungs raises legitimate concerns about: 1) the safety of use of allogeneic grafts; 2) the viability of the graft after administration (regardless of its route); 3) the stability of the cells after administration; 4) the durability of the effects evoked; 5) the effect evoked by repeated administration of the cells. A solution to these problems is to develop a biological product enabling to reproduce the properties of the MSCs, while simultaneously omitting the doubts resulting from the use of whole cells and increasing the repeatability of the subsequent production batches.

Secretory microvesicles (MVs), constituting an element of the secretome of the MSCs, have been known for a dozen or so years. The microvesicles are one of the kinds of secretory extracellular vesicles (EVs) which are differentiated due to the diameter of the individual structures and the process of their formation. In accordance with this concept, the EVs are divided into exosomes (30-100 nm) and microvesicles (100 nm-1 μm) which are formed as a result of the detachment of cell membrane fragments (exocytosis), and apoptotic bodies (1-5 μm) which are produced by cells undergoing apoptosis. A use of the aforementioned structures has been described for the treatment of inflammatory diseases, in the repair of damaged tissues, in the treatment of liver diseases, kidney diseases, cardiovascular diseases and neurological disorders (Shahir M et al., 2020).

The secretory microvesicles (MVs) are well-known and described spherical structures with a diameter of less than 1 μm, having a bilayer lipid membrane, which are released by the cells into the intercellular space under physiological and pathological conditions. These structures can reflect the effects of action of the cells from which they are derived and therefore, a use thereof appears to be an interesting alternative to the use of the preparations based on the utilization of whole cells. The utilization of the MVs may allow to avoid some of the problems related to the implementation of cell therapy (the safety of use, viability, the manner of storage).

The cultures from which a preparation of MVs is to be obtained may be conducted in a targeted manner (in order to produce the preparation) or the preparation may be produced from the reject (a used-up culture medium) emerging after the culturing of these cells.

Methods of isolating vesicles from the MSCs are known in the prior art which are described, for example, in a publication by Müzes G, Sipos F., 2022. A fact is also known that the mesenchymal cells from the human adipose tissue and the vesicles secreted therefrom act on the inflammation and the mechanics of the lungs in an experimental model of allergic asthma (de Castro L.L. et al., 2017). In this publication, stem cells and extracellular vesicles were isolated and the efficacy of both MSC-ATs and EVs was described in decreasing the number of eosinophils in the lung tissue and bronchoalveolar lavages (BALs) and modulating the respiratory tract remodeling, whereby their influence on the T lymphocytes differed in the lungs and thymus. The authors stated that the EVs could be a promising treatment for asthma, however, further studies and clarification of the different mechanisms of action of the AD-MSCs compared to their EVs are required.

Application WO2022223767 A1 discloses a method of isolating adipose tissue-derived EVs from MSCs, wherein the cells for isolating the vesicles were cultured in the presence of extracellular matrix proteins, such as laminins alpha-5, alpha-4 or functional fragments thereof, or in the presence of polypeptides containing the extracellular domain of the human MCAM protein.

In a publication by B.A. et al., 2021, on the other hand, the functional importance of the EV vesicles is described in asthma, one of the most common chronic non-infectious diseases. Mechanistic studies are indicative of the importance of the different subtypes of the EV vesicles and their variable charges in the functioning of the pathways underlying asthma and are indicative of a certain interesting potential for the development of future therapeutic interventions. In turn, associative studies exhibit a good diagnostic potential of the EVs in asthma. It is known from the publication that the immunosuppressive effect of the MSCs is mediated by a number of immunosuppressive mediators, such as NO, IDO, prostaglandin E2(PGE2), TNF-stimulated gene 6 protein (TSG-6), CCL-2 or PD-L1. Since the MSCs must be activated to increase the expression of these therapeutic agents by inflammatory cytokines, such as TNF-α, IFN-γ or IL-1 (Lee et al., 2009a; Wei et al., 2013), the EVs isolated from non-activated MSCs are likely to express lower levels of the therapeutic agents.

Application US20190060368 A1 discloses a pharmaceutically active preparation of extracellular EV vesicles derived from a selected population of activated mesenchymal cells which have an increased TSG-6 level. Wherein the cell population is cultured in a serum-free substrate. The MSCs were incubated in a chemically defined protein-free nutrient medium which activates the MSCs in order to increase therapeutic proteins, including TSG-6, as well as provides a stable environment for producing the EVs. Therefore, specialized preparations of the EVs derived from the MSCs, as produced by the manner described in the application, have an advantage over the EVs produced by the non-activated MSCs. It is also contemplated that the MSCs cultured in the serum-free nutrient medium would be a useful clinical-grade therapeutic product.

The present invention describes a method of obtaining a preparation of MVs from an MSC culture medium and is indicative of the effectiveness of the product in preclinical studies (using a commonly recognized animal model of lower respiratory tract inflammation) for the treatment of asthma and other chronic lower respiratory tract diseases.

The product of the secretory extracellular vesicles derived from the MSCs according to the present invention allows to achieve the object of developing a biological product enabling to reproduce the properties of the MSCs, while simultaneously omitting the doubts resulting from the use of whole cells and increasing the repeatability of the subsequent production batches.

The secretion of small, membraned structures referred to as the secretory extracellular vesicles is one of the mechanisms of secretory action of the MSCs. These structures may contain compounds also contained in the whole cells, namely bioactive lipids, proteins and short non-coding RNA fragments. In connection with this, it appears that the vesicles should mimic the action of the whole MSC cells. The microvesicles, similarly to the cells isolated from different sources and from different donors, are marked by heterogeneity and variability which depend on, among others, the level of activation of the cells producing them, as related to, among others, the condition of the donor. To solve this problem in our studies, the MSCs were subjected to a priming process (stimulation under in vitro conditions) employing a mixture of pro-inflammatory cytokines containing strictly specified concentrations of IL-1β, TNF and IFNγ. This process is a step of the production of a new biological preparation and through use thereof, the problems related to the effectiveness of the process and the heterogeneity of the product are eliminated. The studies presented in the present application indicate that the use of the mixture exhibits an advantage over utilizing the cytokines alone or simultaneously employing only two of these proteins and has not been known thus far. Both IL-1βand TNF cause the production of IL-10 (secretory mechanism), while IFNγ increases the expression of the PD-LI molecule on the cell surface (ligand-receptor mechanism). The use of the mixture of cytokines enables to yield the effect of the final product which most closely reflects the action of whole cells (which thus far has been unachievable).

The proposed innovation may thus constitute a therapeutic alternative for patients with an ineffectively controlled inflammation or who do not respond to the available therapies.

Moreover, based on literature data and bearing in mind the understanding that the microvesicles should reproduce the immunosuppressive activities of the mesenchymal stem cells, the preparation of secretory microvesicles can also find subsequent use in other inflammatory lower respiratory tract diseases with known immune mechanisms, such as, e.g., chronic obstructive pulmonary disease, COVID-19 and idiopathic pulmonary fibrosis. The effectiveness of the preparation of MVs was confirmed in an experimental model of lower respiratory tract inflammation with a predominance of neutrophilic infiltration. Even though the etiology of these respiratory system diseases appears to be dissimilar, certain processes leading to the development of an inflammatory cascade, the composition of the inflammatory infiltration, the cytokine profile and the changes effected by an insufficient control of these processes (fibrosis/remodeling), leading to a limitation of the respiratory function, are common to the indicated entities. The preclinical studies to date have confirmed that the administration of the MSCs affords measurable benefits in terms of reducing inflammation in the respiratory diseases. Based on the literature data and the results of the present authors' own studies, a concept may be adopted that the secretory microvesicles reproducing the effectiveness of whole cells can achieve the desired therapeutic effect.

The object of the present invention is to provide a method of producing a preparation of secretory microvesicles from stem cells and the preparation of microvesicles, as produced by this method, which will find use in therapy in patients with lower respiratory tract inflammation, including severe steroid-resistant asthma, in whom the available treatment is ineffective. It also applies to other respiratory system diseases which progress with a predominance of leukocyte infiltration within the lower respiratory tract (with the Th2-independent inflammation). It is worth emphasizing that the treatment of patients suffering from severe asthma is a major pharmacoeconomic problem in many middle-and/or highly-developed countries, consuming about half of the funds provided for the treatment of asthma overall. The use of the secretory microvesicles solves the technical problems resulting from the use of the whole MSC cells, including the safety of use, viability, and the manner of storage, while maintaining a comparable therapeutic effectiveness.

The method of producing a preparation of secretory microvesicles from mesenchymal stem cells (MSCs), according to the present invention, comprises the following steps:
a) a tissue previously isolated from the human body is subjected to, preferably mechanical, fragmentation,
b) the tissue fragmented in step a) is digested enzymatically, whereupon optionally the enzyme is inactivated,
c) large tissue fragments are separated, and then the remaining cell suspension is centrifuged, preferably at a speed of 300-1000×g at a time of 2-15 minutes, most preferably at 400×g for 5 minutes, at room temperature to obtain a pellet of MSC cells,
d) the MSC cells obtained in step c) are cultured in an MSC-dedicated culture medium supplemented with an antibiotic, wherein the cells are subjected to, at least triple, passaging at a temperature of 34-39 °C, preferably of 37 °C,
e) the MSC cells obtained as a result of step d) are cultured in the MSC-dedicated culture medium with the addition of at least one human cytokine selected from a group including IL-16, TNFα, IFNγ, and mixtures thereof at a temperature of 34-39 °C, preferably at 37 °C,
f) the culture medium with the MSC cells from step e) is pre-purified by pre-centrifugation at 300×g-500×g for at least 3 minutes, preferably for 10 minutes, at room temperature, and then by subsequent centrifugation at a speed comprised in the range of 1000×g-3000×g for at least 3 minutes, preferably for 10 minutes, at room temperature, to obtain a supernatant of the culture medium, containing the secretory microvesicles,
g) optionally, the supernatant of the culture medium, containing the secretory microvesicles, from step f) is frozen at a temperature of −20 °C or lower for the purpose of storage, and after a desired amount of time, the culture medium is defrozen,
h) the supernatant of the culture medium, containing the secretory microvesicles, is subjected to at least one ultracentrifugation to obtain the preparation of secretory microvesicles in the form of a precipitate.

Preferably, in step a), the fragmentation is performed by cutting.

Preferably, in step b), the enzyme is a collagenase, preferably collagenase I or collagenase IV or collagenase A or collagenase VII, or another enzyme with proteolytic activity, preferably a trypsin or a hyaluronidase or a serine protease.

Preferably, in step b), collagenase IV, preferably at a concentration of 1 mg/mL, is used as the enzyme, wherein the fatty tissue with collagenase IV is subjected to incubation for at least 45 minutes at 37 °C, under an atmosphere of 5% CO₂ saturation with continuous mixing.

Preferably, collagenase IV is inactivated by adding an inactivating agent in a ratio of 1:1 relative to the volume of collagenase IV, wherein the inactivating agent comprises a DMEM medium.

Preferably, step c) the large tissue fragments are separated by filtration, preferably using a filter with pores of from 40 μm to 100 μm.

Preferably, in step d), the antibiotic is gentamicin.

Preferably, in step d), IL-1βat a concentration of 1-100 ng/ml or TNFα at a concentration of 1-100 ng/ml or IFNγ at a concentration of 1-100 ng/ml or mixtures thereof are used as the human cytokine, wherein the culturing in step d) is conducted for at least 72 h, preferably for 96 h.

Preferably, in step d), IL-1βat a concentration of 25 ng/ml or TNFα at a concentration of 50 ng/mL or IFNγ at a concentration of 50 ng/ml or mixtures thereof are used as the human cytokine.

Preferably, in step d), a mixture comprising IL-1β at a concentration of 25 ng/mL, TNFα at a concentration of 50 ng/ml and IFNγ at a concentration of 50 ng/ml is used as the human cytokine.

Preferably, in step f), the culture medium is subjected to pre-centrifugation at room temperature at a speed of 300×g for 10 minutes, and then to a subsequent centrifugation at room temperature at a speed of 2000×g for 10 minutes.

Preferably, in step h), the culture medium is subjected to ultracentrifugation, preferably at 100,000×g for 60 minutes at 4 °C, to obtain the microvesicles in the form of a pellet, wherein the ultracentrifugation is repeated, and after the first ultracentrifugation, the sediment is rinsed, preferably with a concentrated buffered saline solution.

Preferably, after step h), the precipitate of microvesicles is suspended in the buffered saline solution, whereupon optionally the suspension is frozen at −80 °C.

Preferably, the tissue is adipose tissue which is derived from fatty skin folds taken from a patient after an abdominoplasty procedure, wherein the patient is preferably a bariatric patient.

Preferably, the tissue is derived from Wharton's jelly.

The room temperature is assumed to be a temperature from 15 to 25 °C, optimally of about 20 °C.

The ultracentrifugation should be understood as a procedure the purpose of which is to increase the centrifugal force (by increasing the rotational speed at a minimum of 70,000×g, optimally 100,000×g) and extending the centrifugation time (a minimum of 30 minutes, optimally for 1 hour) enabling the separation and formulation of a pellet from structures with a size of less than 1 μm. Due to their small weight and volume, the microvesicles are subject to little centrifugal force under standard centrifugation conditions. In connection with the above, the effective separation of this fraction in the form of a pellet requires an increase in centrifugal force and an extension of the procedure time under given temperature conditions (optimally at 4 °C)

The preparation of secretory microvesicles of mesenchymal stem cells, as produced by the method of the present invention, finds use as a drug.

The preparation of secretory microvesicles of mesenchymal stem cells, according to the present invention, finds use in the treatment of inflammatory lower respiratory tract diseases, preferably chronic obstructive pulmonary disease or COVID-19 or idiopathic pulmonary fibrosis or severe asthma.

Incubation of the MSCs in an in vitro culture with at least one of the indicated cytokines and/or a mixture thereof is for the purpose of increasing the effectiveness of the preparation of the microvesicles secreted by these cells before they undergo further processing (centrifugation, ultracentrifugation) and subsequent administration into the lower respiratory tract. The preparation of the microvesicles derived from the cytokine-stimulated MSCs was experimentally tested and exhibited distinctive differences in terms of immunosuppressive activity compared to the preparation derived from a non-stimulated culture produced by the same method. The present invention can aid in, among others, the development of a new therapy for patients with severe and/or steroid-resistant asthma who are far more often subjected to hospitalization, and the expenses for their treatment constituting a major pharmacoeconomic problem in the middle-and highly developed countries due to the lack of effective methods.

### EXPLANATION OF THE DRAWINGS

FIG. 1. A representative cytometric analysis of the expression of MSC-characteristic markers on the surface of MVs (CD73, CD90, CD105). The upper panel shows gating based on calibration beads of a known size that enables to restriction of the analysis only to structures smaller than 1 μm. The gating for the individual markers was carried out employing an unstained sample of MVs (the middle panel). A CD73 FITC-, CD90 PE-and CD105 APC-stained sample of MVs is presented in the bottom panel.

FIG. 2. A representative cytometric analysis of the proliferation of CFSE-stained CD4+ lymphocytes stimulated with anti-CD3/CD28 in the presence of specific doses of MVs or in the absence thereof. The lymphocytes were determined to be cells of a medium size and a low granularity. Then, cells with expression of CD4 (a marker found on the surface of the helper T lymphocytes) were gated. The histograms show the individual populations of the proliferating cells (p0, p1, p2). Non-mitogen-stimulated cells constituted the control for the study.

FIG. 3. The levels of cytokines and a soluble form of the CD163 protein after stimulation of PBMCs with LPS in the presence of MVs or in the absence thereof (Veh).

FIG. 4A Graphical summary of the experimental model of neutrophilic asthma and 4B a diagram of the division of a lung, utilized for the purpose of carrying out the individual analyses.

FIG. 5 Representative photographs of haematoxylin and eosin (H&E)-stained lungs of mice (n=5) 5A. A summary of cytometric analyses of the frequencies of populations of IL-4-, IFN-γ and IL-10-producing effector T lymphocytes (n=5) 5B. The results are presented as means with a standard deviation. A static analysis was performed employing the ANOVA test with the Fischer's test (post-hoc) employing the GraphPad Prism v9 program, *p<0.05; **p<0.01.

FIG. 6 Changes in levels of A. interleukins and GM-CSFs as well as chemokines B. CCLs and C. CXCLs. Analysis of the level of analytes was performed employing the multiplex method in samples of BALs after administration of the preparation of MVs. The differences between the studied groups were analyzed employing the ANOVA test with the Fisher's test (post-hoc) in GraphPad Prism v9; *p<0.05; **p<0.01; ***p<0.001; ****p<0.0001.

FIG. 7 A. Graphical summary of a long-term experimental model of asthma and 7 B. the changes caused by the administration of the preparation of MVs in an inflammatory infiltrate. Representative photographs of the hematoxylin and eosin (H&E)-stained lungs of mice (n=5) and C. changes in the frequencies of the IFN-γ, IL-4-, IL-17-, and IL-10-producing effector T lymphocytes (n=5). The results are presented as means with a standard deviation. The static analysis was performed employing the ANOVA test with the Fischer's test (post-hoc) employing the GraphPad Prism v9 program, *p<0.05; **p<0.01

FIG. 8 Changes in the expression of genes associated with A. the proliferation of lymphocytes, B. the migration of leukocytes, C. eicosanoids and lipid mediators, D. prostaglandin synthetases after the administration of MSCs (HDM+MSC), of the vesicles derived from a non-stimulated (HDM+MVs) and a stimulated culture (HDM+pr-MVs). All the results were presented as log2FC (log2foldchange) against an animal group stimulated with a house dust mite (HDM) extract and not receiving any of the preparations. The differences between gene expression in the studied groups were assessed employing the Wald test with Benjamini-Hochberg correction for multiple testing. ****padj<0.0001; ***padj<0.001; **padj<0.01; *padj<0.05.

### EMBODIMENTS

The mesenchymal stem cells used in the method according to the present invention were derived from an adipose tissue taken from fatty skin folds of a patient qualified for an abdominoplasty procedure. The adipose tissue was previously taken from the removed fold directly in the operating room or in a properly prepared laboratory under sterile conditions. The taken tissue was subjected to further processing.

### Example 1

### Preparation of an MSC Culture (Isolation of MSC and Stimulation Thereof With a Cytokine Mixture).

In an initial procedure, the adipose tissue (in an amount corresponding to about 20-25 mL) is subjected to mechanical fragmentation (cutting into fragments with sizes of about 0.5 cm×0.5 cm). Then, for the purpose of degrading the collagen fibrils found in the connective tissue, collagenase IV with an activity of not less than 265 U/mg, at a concentration of 1 mg/mL (diluted in 15 mL of a single-concentrated phosphate-buffered saline solution, 1×PBS), as per the information from the manufacturer, is added to the comminuted tissue. The fatty tissue with collagenase IV is subjected to 45-minute incubation under optimal culture conditions (37 °C, under an atmosphere of 5% CO₂ saturation) in accordance with the instructions of the manufacturer of collagenase IV. Moreover, in order to increase the effectiveness of the enzyme's action, the tissue together with the buffer is subjected to constant mixing throughout the duration of the incubation. The action of the enzyme is subjected to inactivation by adding an inactivating agent in the form of a culture medium, i.e., DMEM +10% FBS (minim. in a ratio of 1:1 relative to the volume of the added enzyme). Alternatively, instead of collagenase IV, another collagenase (e.g., collagenase I, collagenase A, collagenase VII) or another enzyme with proteolytic activity (e.g., a trypsin, a hyaluronidase, serine proteases) may be used, and to inactivate the enzyme, another animal or non-animal inactivating agent may be used.

The larger adipose tissue fragments are removed from the resulting suspension on a filter with a pore diameter of from 40 μm to 100 μm, preferably of 100 μm, to allow optimal separation of the MSCs (reaching a diameter of even up to 30 μm) from the tissue fragments, including also the fragmented adipose tissue. The sample is centrifuged at a speed of 400×g for 5 minutes at a temperature of 20 °C. The isolated cells are cultured at a temperature of 37 °C in a dedicated medium promoting their growth and proliferation—the Mesenchymal Stem Cell Basal Medium for Adipose, Umbilical and Bone Marrow-derived MSCs—with the addition of the Mesenchymal Stem Cell Growth Kit for Adipose and Umbilical-derived MSCs—Low Serum, containing rh FGF-basic: 5 ng/ml; rh FGF-acidic: 5 ng/ml; rh EGF: 5 ng/ml; Fetal Bovine Serum FBS: 2%; L-Alanyl-L-Glutamine: (2.4 mM) with the addition of gentamicin (Gibco, 50 mg/mL) at a concentration of 10 μg/mL per culture. Other suitable dedicated culture media for the MSC cells are, e.g.,: a) the Mesenchymal Stem Cell Expansion Medium (1×), SCM015, Sigma Aldrich; b) the Mesenchymal Stem Cell Growth Medium 2, C-28009, PromoCell; c) the MSC NutriStem® XF Basal Medium, without phenol red, 05-202-1A and the MSC NutriStem ® XF Medium, 05-200-1A, Sartorius; d) the MesenCult™ Mesenchymal Stromal Cell Culture, 05401 and the MesenCult™-ACF Plus Medium, 05448, Stem Cell Technologies; e) the StemPro™ MSC SF, A1033201 and the StemPro™ MSC SFM XenoFree, A1067501, Gibco, ThermoFischer; f) the Mesenpan Special Medium for Human Mesenchymal Stem Cells, w/o: FBS, P08-50400K, PAN Biotech; or their equivalents. Instead of the addition of gentamicin, other antibacterial and/or antifungal agents optimized for given culture types and the employed culture media, e.g. penicillin, streptomycin, amphotericin B, may be used.

Alternatively, the cells may be cultured at a temperature of between 34 and 38 °C, wherein 37 °C is preferable. The moment of isolation is counted as the zero passage. In each passage, the MSCs cells overgrow to a confluency level of 80% within a time of about 1-1.5 weeks.

The phenotype and the properties of the MSCs are confirmed after the 3rd passage in accordance with the minimum criteria adopted by the International Society for Cell & Gene Therapy. Continuous passaging and the introduction of the cells into higher passage numbers may change their phenotype and functionality and not afford such advantages as the passage 3. After the 3rd passage, the cells are cultured in the aforesaid dedicated medium, but without the addition of the antibiotics, alone or in the presence of a mixture of recombinant human cytokines IL-1βα (25 ng/ml), TNF (50 ng/ml), IFNγ (50 ng/ml) for 96 h, optimally at 37 °C (alternatively 34 °C-39 °C). Alternatively, one or more of the aforementioned cytokines may be used. After the process, the medium is collected into aseptic tubes. The medium is subjected to pre-centrifugation at a speed of 300×g for 10 minutes, and then of 2000×g for 10 minutes. Both centrifugations are performed at room temperature. The prepared samples of the culture medium collected as a supernatant after the centrifugation are frozen in aseptic tubes at −20 °C (up to 2 weeks), −80 °C (up to 6 months) or below −125 °C (up to more than a year).

### Example 2

### Obtaining of a Preparation of MVs (Isolation of the Microvesicles From the Culture Medium)

The culture medium of example 1 is defrozen at a temperature of 37 °C, and then transferred into special aseptic tubes designed for ultracentrifugation. Each time, it is possible to centrifuge ca. 40 mL of the medium. In order to attain a preparation of microvesicles which is as thickened as possible and purified of the components of the medium, 2 ultracentrifugations are taken into consideration in the procedure, both under the conditions of: 100,000×g, 60 minutes, 4 °C. After the first centrifugation, the pellets are rinsed with a single-concentrated buffered saline solution (a 5-mL PBS). The concentration of total protein is assayed in the preparation, which constitutes a reference to the amount of the microvesicles obtained. To the precipitate of microvesicles, as obtained from the centrifugation of 7 tubes of the medium, 50 μL of a buffered saline solution (PBS) are added. Alternatively, a physiological saline solution or a single-concentrated PBS (a body-neutral solution, including various variants of saline) may be added. The suspension (the preparation of microvesicles) is frozen at −80 °C. To the precipitate of vesicles from one tube, 50 μL of a RIPA buffer with protease inhibitors in the form of an extract of pancreatic enzymes inhibiting the activity of metalloprotease, chymotrypsin, trypsin, papain and the so-called pancreatic extract are added and frozen at −80 °C. In this preparation, the content of protein is studied. In accordance with the literature data available, 100 μg of a protein from the preparation of microvesicles is assumed to correspond to 5×10⁵ MSC cells.

### Example 3

### Evaluation of the Potential of the Microvesicles to Limit Respiratory Tract Inflammation.

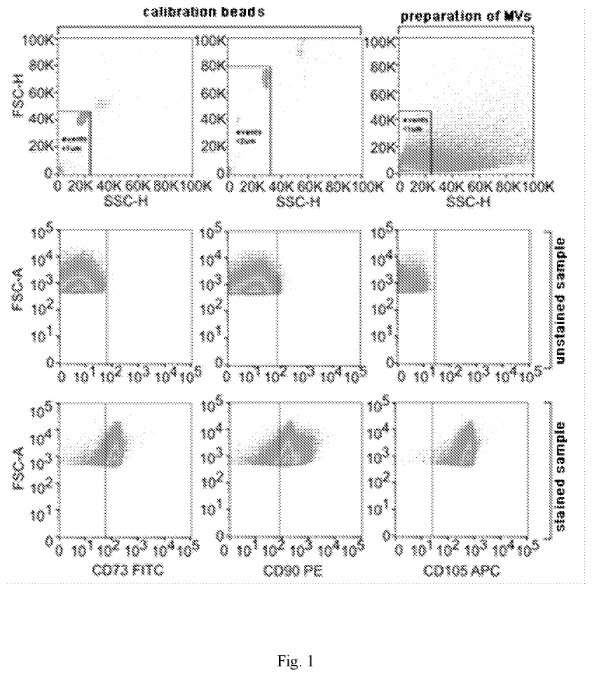

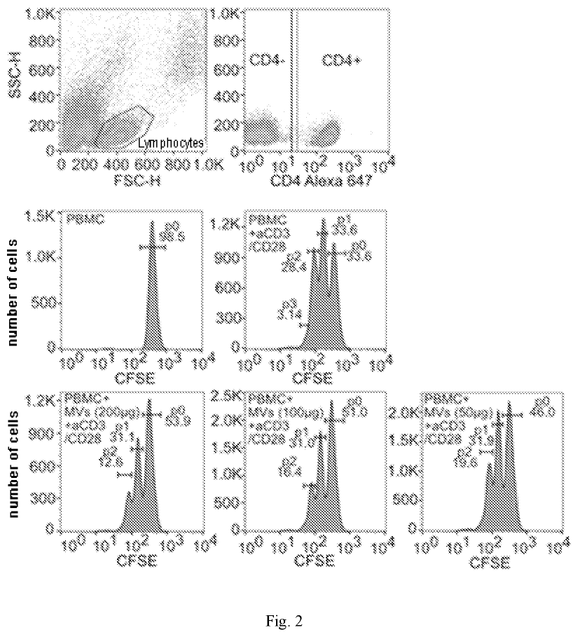

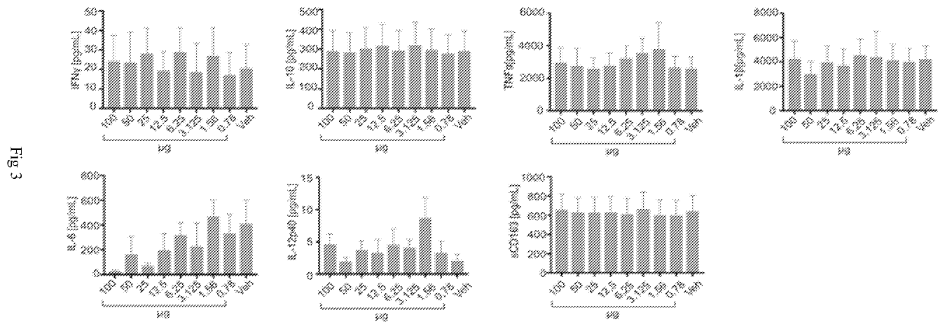

The effectiveness of the present invention was confirmed on in vitro and in vivo preclinical models. The yielded preparation of microvesicles was subjected to experimental studies utilizing commonly accepted in vitro as well as in vivo pre-clinical models for the purpose of demonstrating the effectiveness of action. In the first step, the expression of MSC-characteristic markers on the surfaces of the isolated MVs (FIG. 1), immunosuppressive activities of the MVs in a T lymphocyte proliferation test (FIG. 2) and the immunomodulating activities on the basis of the level of cytokines after stimulation of peripheral blood mononuclear cells (PBMCs) with a lipopolysaccharide (LPS) of the E. coli bacteria (FIG. 3) were studied.

The analysis of the phenotype of the MVs was performed by the MoFlo Astrios EQ cell sorter (Beckman Coulter) which enables to assess very small events (elements, structures). In accordance with the definition, cellular microvesicles are structures with a diameter of no more than 1 μm. For the purpose of performing the analysis, calibration beads with a known size were employed, which enabled to restriction of cytometric analysis down only to structures with a diameter corresponding to the size of the microvesicles. In this manner, it was confirmed that the microvesicles yielded by the method according to the present invention exhibit the expression of the MSC-characteristic markers, namely CD73, CD90 and CD105 (FIG. 1). Moreover, a limitation of the proliferation of T lymphocytes stimulated with mitogens (antibodies directed against CD3/CD28 surface molecules allowing for the activation of both receptors-these antibodies are commonly utilized as a mitogen for T lymphocytes) was observed in the presence of the MVs compared to cells which had not been incubated with the preparation. Lymphocytes not subjected to the stimulation constituted the control for the study (FIG. 2). A reduction of the proliferation of T lymphocytes was observed in the presence of the MVs. In the last step of the in vitro studies, the levels of cytokines (IFNγ, IL-10, TNFα, IL-1β, IL-6, IL-12p40) in the culture supernatant as well as the soluble form of the CD163 protein (sCD163), as secreted by the PBMCs stimulated for 24 h with LPS at a concentration of 1 μg/mL, were assessed in the presence of selected concentrations/amounts of the MVs (0.78 μg; 1.56 μg; 3.125 μg; 6.25 μg; 12.5 μg; 25 μg; 50 μg; 100 μg) or in the absence thereof (veh). No changes in the level of the secreted cytokines were observed under the selected culture conditions. The results of the preclinical studies conducted so far have been clearly indicative of a high potential to reduce inflammation associated with the activity of T lymphocytes.

### Example 4

### Evaluation of a Preparation Containing the Microvesicles in a Short-Term Experimental Model of Lower Respiratory Tract Inflammation.

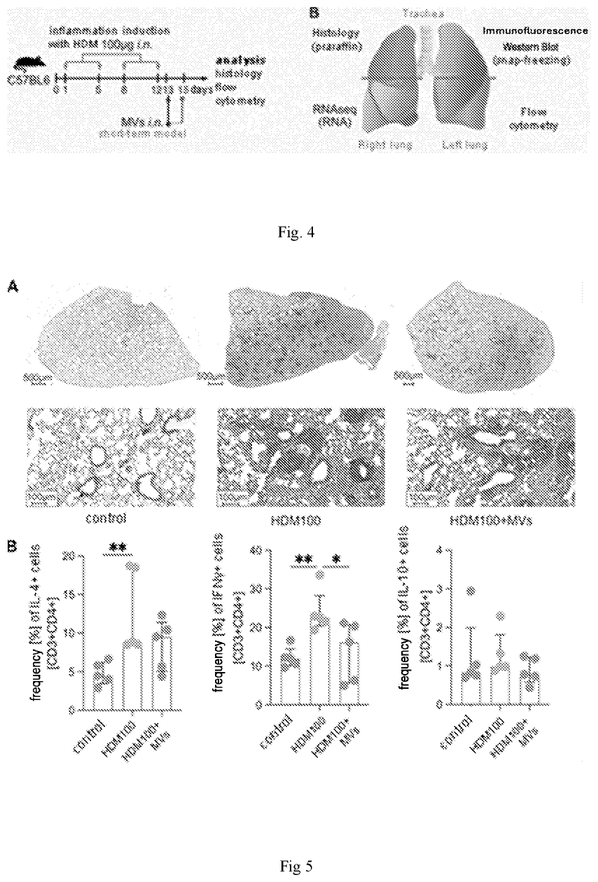

The results of the in vitro studies confirmed that the procedure utilized enables to yield a preparation containing the microvesicles with the required phenotypic features. In connection with the above, in the next step, the therapeutic effectiveness of the preparation was assessed in an experimental mouse model of house dust mite (HDM) extract-induced airway inflammation occurring in humans. Mice of the C57BL6 strain (females, at the age of 6-8 weeks) were utilized in the experiments. The study model utilized in the present invention enables a faithful reproduction of the features of neutrophilic asthma occurring in humans (FIG. 4A).

For the purpose of inducing neutrophilic lower respiratory tract inflammation, the HDM extract was administered intranasally to the mice at a dose of 100 μg for 5 consecutive days (over two weeks) which were followed by two days of rest (FIG. 4A). The microvesicles derived from the medium after the culturing of the MSCs were also administered intranasally to the animals on the 13th day of the experiment. Animals with induced airway inflammation were utilized as the control which were administered with the preparation after centrifugation of the culture medium in which there were no cells. Before the process of centrifugation, the medium was subjected to the identical procedures as in the case of the medium after the culturing of the MSCs (several day-long storage of the medium under standard incubation conditions, freezings, centrifugation). This approach enabled inventors of said invention to assess whether the observed therapeutic effect is characteristic of the preparation with the MVs, or whether it only constitutes the effect of the components contained in the culture medium. Additionally, mice were incorporated into the experiment receiving only a 0.9% saline solution (at the analogous time points as for the HDM extract-solvent) and a pure culture medium on the 13th day of the experiment. All the mice were sacrificed on the 15th day of the study. Bronchoalveolar lavages (BALs) were collected from each mouse for the purpose of assessing the level of cytokines, chemokines and growth factors as well as the lung divided acc. to the scheme attached in FIG. 4B. In order to maintain the maximum repeatability of the tests, the lungs were always divided acc. to the adopted scheme and secured in accordance with optimized procedures, namely for the purpose of:
carrying out histochemical stainings, the pulmonary lobes were fixed in a paraformaldehyde solution, and then immersed in paraffin after 24 hours;
assessing the frequencies of the effector T lymphocytes, the lungs were stored in an RPMI 1640 medium containing a 10% FBS until the initiation of the cell dissociation procedure (an increase in the viability of the cells);
carrying out an assessment of the changes in the expression of genes (qPCR-targeted analyses or transcriptomic analyses), the lungs were stored for a period of 72 h from the time the tissues in the RNA solution were taken, which enabled to stabilize the cellular RNA (the analyses described in the present section are the basis for the subsequent studies);
perform targeted analyses (e.g. immunofluorescence stainings, Western Blot), the tissue was instantly cooled by immersion in liquid nitrogen (snap-frozen), enabling the maintain the structure of the sample (the targeted analyses described in the present section are the basis for the subsequent studies performed on the basis of the changes described in the transcriptome after the administration of the MVs).

In order to assess the infiltration of the immune cells within the lung tissue, haematoxylin and eosin (H&E) staining was carried out acc. to the standard procedure, employing paraffin-immersed cuttings. Acid-absorbent structures (the cytoplasm) were stained with eosin, whereas alkaline-absorbent structures (the cell nuclei) were stained with hematoxylin (FIG. 5A). The presented photographs were made by the Pannoramic 250 FLASH III histological preparation scanner. It was observed that administrating the mice with the HDM extract at a dose of 100 μg causes the development of lung inflammation (HDM100, the middle panel of FIG. 5A), which was not noted in the case of administrating saline (the control, the left panel of FIG. 5A). Administration of the MVs limits the inflammatory infiltration in the mice in an experimental model of neutrophilic asthma (HDM100+MVs, right panel FIG. 5A). It is worth mentioning that this effect was not apparent in the case of administering the animals with a preparation from the culture medium, i.e. one not containing the MVs (HDM100, the middle panel of FIG. 5A). These studies confirm the therapeutic effect of the new preparation, consisting in inhibiting inflammation within the lower respiratory tract.

In the next step, cytometric analysis was carried out of the cellular elements associated with inflammation within the lungs. The frequencies were assessed of the helper T lymphocytes (CD3+CD4+) producing IL-4, IFNγ and anti-inflammatory IL-10, respectively (FIG. 5B). In order to yield a homogeneous suspension of the cells, the lungs were dissociated utilizing a mechanical-enzymatic method employing the Lung Dissociation Kit (Miltenyi Biotec) in accordance with the procedure attached by the manufacturer. Then, the cells were stimulated for 6 h with a leukocyte-activating cocktail (PMA-IO) with a protein transport inhibitor (GolgiPlug, Brefeldin A) (BD Biosciences). Stimulation of the cells by means of this cocktail causes the effector cells to produce cytokines, while simultaneously blocking their secretion (accumulation of the cytokines inside the cells). Such an approach enables to assessment of the population of the T cell effectors shaping the inflammatory microenvironment within the analyzed tissue. In the next step, ex vivo-stimulated cells were incubated with fluorochrome-coupled antibodies (anti-CD45, anti-CD3, anti-CD4) for a period of 30 minutes without access to visible light (extracellular staining). Then, the samples were subjected to the process of permeabilization for the purpose of facilitating the penetration of the antibodies into the interior of the cell. The cells were subjected to intracellular staining with fluorochrome-coupled antibodies (anti-IL-4; anti-IL-17A, anti-IFNγ, anti-IL-10) under the same incubation conditions. The samples were analyzed employing the FACSCanto flow cytometer (BD Biosciences). The yielded data was analyzed utilizing the FlowJO software (BD Bioscences). As predicted, an increase in the abundance of the population of IL-4-(5-10%) and IFNγ-producing (ca. 10%) lymphocytes was noted in the mice with induced lower respiratory tract inflammation compared to the control mice. Administration of the MVs caused a decrease in the frequency of the IFNγ-producing T lymphocytes (a subpopulation of Th1) compared to the animal group not receiving the MVs. This effect was not apparent in the IL-4-producing T lymphocytes (a subpopulation of the Th2 lymphocytes). Interestingly, no statistically significant changes were observed in the abundance of the population of the anti-inflammatory IL-10-producing T lymphocytes in any of the studied groups.

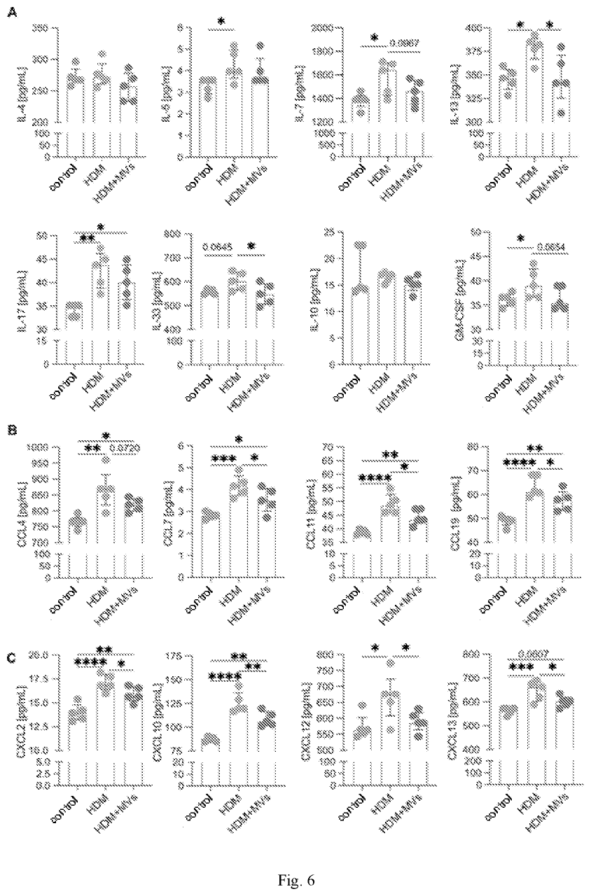

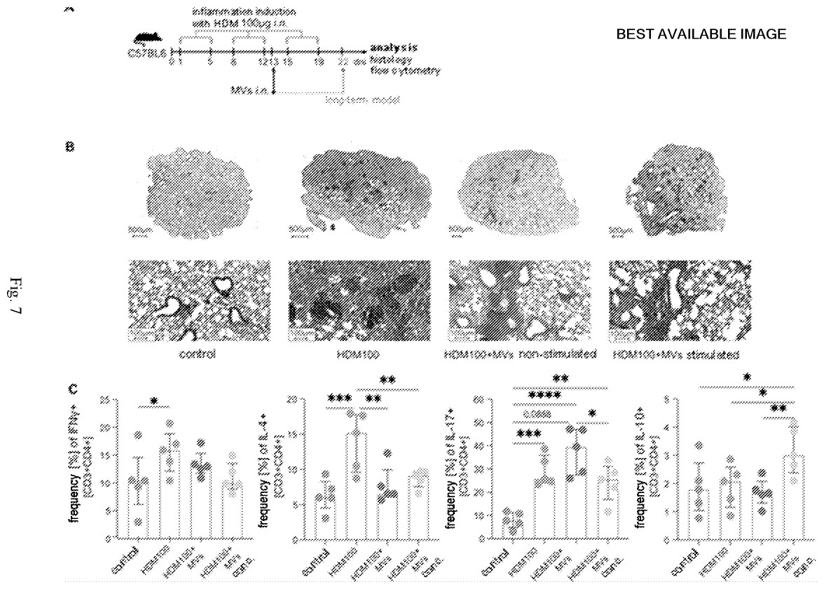

Then, the changes were assessed in the levels of protein analytes (cytokines, chemokines and growth factors) in the BALs collected from mice. The samples of the BALs were collected by introducing a buffered saline solution (Corning) together with protease inhibitors (complete Mini Tablets, Roche) into the respiratory tract and then aspirated by means of a sterile syringe. The evaluation of the level of selected protein analytes, which are of importance during the development of lower respiratory tract inflammation, was carried out employing the Luminex kit. This test enables a quantitative evaluation of many proteins in a relatively small volume of the material under study. As expected, elevated levels of IL-5, IL-7, IL-13, IL-17 and the granulocyte and macrophage colony-stimulating factor (GM-CSF) as well as a marked trend towards an increase in the levels of IL-33 were observed in the mice with induced lower respiratory tract inflammation (FIG. 6A). Administration of the preparation of MVs caused a decrease in the level of the Th-driven cytokines, namely IL-13-and IL-33 by ca. 50 pg/mL and a marked trend towards a reduction of the level of IL-7 (by ca. 50-100 pg/mL) compared to the animals not receiving the preparation. Moreover, there was a statistically significant decrease in the concentration of GM-CSF which plays a significant role in the activation and migration of eosinophils, neutrophils, and monocytes/macrophages. Administration of the MVs did not cause changes in the levels of IL-4, IL-17, and the anti-inflammatory IL-10 compared to the mice that did not receive the MVs. The induction of neutrophilic lower respiratory tract inflammation was also characterized by an increase in the level of some chemokines belonging to the CCL (FIG. 6B) and CXCL families (FIG. 6C). Administering the animals with the HDM extract caused an elevation of the concentrations of CCL4, CCL7, CCL11 and CCL19 (FIG. 6B) and CXCL2, CXCL10, CXCL12 and CXCL13 (FIG. 6C) compared to the control mice receiving the saline solution. The use of the preparation of MVs resulted in, on the other hand, a statistically significant reduction of the level of CCL7, which is a chemoattractant for a number of immune cells, including monocytes, eosinophils, basophils, dendritic cells, NK cells and activated T lymphocytes (FIG. 6B). Moreover, a reduction was observed in the level of CCL11 (eotaxin 1), responsible for the migration of eosinophils in allergic response and asthma (FIG. 6B). A lower level of CCL19 was also observed, which is responsible, among others, for the migration of dendritic cells and T lymphocytes (FIG. 6B). The key role of CCL19 and CCL21 (data not presented) in the development of lower respiratory tract inflammation has been documented in the previous studies. Moreover, a trend was also noted (at the borderline of statistical significance) towards a reduction of the level of the CCL4 chemokine after administration of the preparation of MVs (FIG. 6B). Some scientific reports draw attention to the correlation of CCL4 with the number of neutrophils and the development of neutrophilic inflammation, putting CCL4 forward as one of the biomarkers of neutrophilic asthma. Analogously to the results obtained in the case of the chemokines belonging to the CCL family, the administration of the preparation of MVs caused a significant reduction of the levels of the CXCL cytokines, namely CXCL2, CXCL10, CXCL12 and CXCL13 (FIG. 6C). CXCL2 is indicated to play a major role in promoting the migration of smooth muscle cells in the course of asthma. CXCL10, which is secreted in response to a high concentration of IFNγ, is, in turn, implicated in the development of a non-Th2-type inflammation (which progress with a predominance of leukocyte infiltration and is Th1 profile-dependent). Moreover, a reduced level of CXCL12 (also known as SDF-1) has been observed, which may be related to the limited chemotaxis of T lymphocytes. Furthermore, CXCL12 has also been proven to be involved in the process of angiogenesis, which is an important component of remodeling. The multiplex analysis also showed a reduced level of CXCL13, which is associated with the chemotaxis of B lymphocytes, after the administration of the preparation of MVs. The presented data indicate that the administration of the preparation of MVs prepared acc. to the optimized procedure decreases the concentration of many chemokines associated with the promotion of chemotaxis of the immune cells crucial for the development of asthmatic inflammation, which may in turn contribute to the reduction of inflammatory infiltration within the lung tissue.

### Example 5

### Evaluation of the Preparation Containing the Microvesicles in a Long-Term Experimental Model of Neutrophilic Asthma.

The use of the preparation of MVs in a short-term model, i.e. one that evaluates the effectiveness of the product after 48 hours from the moment of administration, allowed to limit lower respiratory tract inflammation. In the subsequent experiment, it was assessed whether this effect could be retained over a longer period while continuing the stimulation of the mice with the HDM extract. At the same time, having found, that the immunosuppressive properties of the MSCs are activated in the presence of pro-inflammatory cytokines, i.e. IL-162 , TNFα, IFNγ, it was also assessed whether the preparation of MVs that is derived from an MSC culture stimulated with the indicated recombinant proteins is characterized by a greater therapeutic potential.

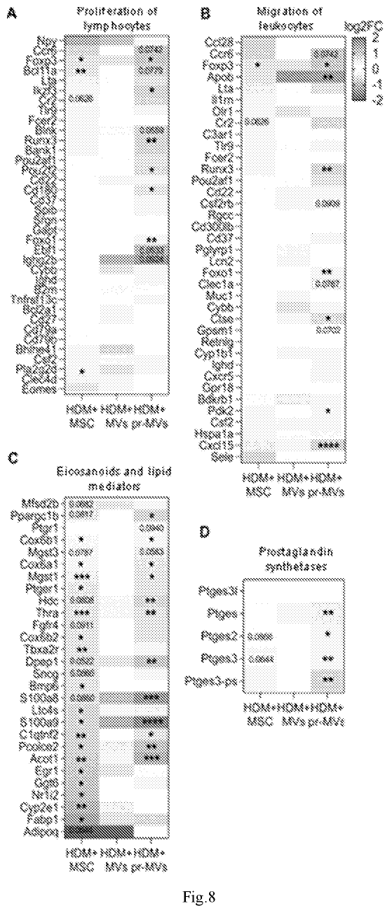

For this purpose, an analogous experimental model of neutrophilic asthma was performed, a graphical summary of which is shown in FIG. 7A. In the model utilized, mice of the C57BL6 strain (females, at the age of 6-8 weeks) were stimulated with the HDM extract at a dose of 100 μg for 5 days in each of the two weeks (inflammation induction). On the 13th day, the mice were administered intranasally with 100 μg of the preparation of MVs (derived from the non-stimulated or stimulated MSC culture). However, after the administration of the product, the stimulation of the mice was prolonged by another week during which the mice continued to be administered with the HDM extract. On the 22nd day of the experiment (72 h since the last administration of HDM), all the mice were sacrificed. Additionally, on the 13th day, one of the groups of mice with induced neutrophilic lower respiratory tract inflammation received a preparation derived from a pure culture medium (analogous to the experiment performed under the short-term model). Animals receiving a saline solution and a product yielded from the pure culture medium constituted the control in the experiment. The BALs and the individual lung lobes were secured in an identical way as in the case of the short-term model, in accordance with the description contained above (FIG. 7B). The methodology involving the H&E staining was performed in accordance with the procedure attached by the manufacturer. The evaluation of the frequencies of the populations of the effector T lymphocytes was performed in accordance with the optimized protocol (as described above, Example 4).

In the first step of this part of the experiment, inflammatory infiltration of the pulmonary tissue was assessed. Analogously to the results of the short-term model, the stimulation of the mice with the HDM extract caused the development of an inflammation within the lung (the second panel, FIG. 7B). Administration of the preparation of the MVs derived from both the non-stimulated (the third panel, FIG. 7B), and the stimulated cultures (the fourth panel, FIG. 7B) allowed to limit inflammatory infiltration compared to the mice not receiving the preparation of MVs, but only a product containing the pure culture medium (the second panel, FIG. 7B). However, in the case of inflammatory infiltration of the pulmonary tissue, no apparent changes were observed in the histological image when using the MVs from the culture stimulated with with the cytokine mixture (the fourth panel, FIG. 7B) compared to the MVs derived from the non-stimulated culture (third panel, FIG. 7B). It should be emphasized, however, that the cellular composition of the infiltration and the expression of proteins in the infiltration were not known.

Then, cytometric analysis was conducted of the frequency of the subpopulations of the effector T lymphocytes producing IFNγ, IL-4, IL-17, and anti-inflammatory IL-10, respectively. In the mice with induced respiratory tract inflammation, an increase in the frequency of the IFN-γ, IL4-and IL-17-producing T lymphocytes was observed compared to the control group, however, no change in the abundance of the population of the IL-10-producing lymphocytes was observed (FIG. 7C). It was observed that the use of the preparation of MVs from the unstimulated culture as well as the stimulated culture leads to a slight reduction of the frequency of the IFNγ-producing cells (FIG. 7C). Additionally, the administration of both preparations of MVs causes a reduction of the abundance of the cells producing IL-4 (FIG. 7C). In the case of the IL-17-producing T lymphocytes, no changes were noted after the administration of the product of MVs compared to the non-treated animals. Interestingly, the frequency of the IL-17-producing T lymphocytes was statistically significantly lower when administering the MVs from the stimulated culture compared to the non-stimulated culture. In the context of inflammatory diseases, the progression of which involves IL-17, the reduction of the abundance of these cells also constitutes a major advantage. The elevation of the frequency of the IL-10-producing T lymphocytes was specific solely and exclusively to the animals treated with the MVs derived from the cytokine-stimulated MSC cells, which constitutes an advantage over the animals receiving the MVs from the non-stimulated culture and over the untreated animals where these changes were not observed.

The transcriptomic analyses of the level of gene transcription in the lung cells showed that both the administration of the MSCs (HDM+MSC), and the vesicles produced by the innovative method (HDM+pr-MV) showed a similar pattern of changes in the expression of genes associated with, among others, the proliferation of lymphocytes (FIG. 8A), the migration of leukocytes (FIG. 8B), eicosanoids and lipid mediators (FIG. 8C) and prostaglandin synthetases (FIG. 8D). These changes differed between the product produced by the innovative method according to the present invention (HDM+pr-MV) to the product obtained by the methods described in the literature (HDM+MV).

To conclude, the conducted experimental verification confirms that the optimized procedure allows to attainment a preparation containing the MVs derived from MSCs isolated from adipose tissue taken from patients after abdominoplasty. The cytometric analysis showed that the MVs attained are characterized by the expression of MSC-characteristic markers. Moreover, the MVs can inhibit the proliferation of the lymphocytes stimulated in vitro with anti-CD3/CD28. The use of the preparation of MVs in the experimental model of asthma confirmed their therapeutic potential. A reduced inflammatory infiltration was observed within the lung tissue, both as a short-term effect of the action of the MVs from the non-stimulated culture and as a long-term effect after the administration of the MVs from both culture types. Some differences in the abundance of the subpopulations of the effector T lymphocytes were observed between the preparations used, which could potentially be indicative of different mechanisms of action of these products.

The production of the preparation (in accordance with the optimized procedure) lasts for 34 weeks.

Moreover, the proposed invention can also find use in other respiratory system diseases (chronic obstructive pulmonary disease (COPD), COVID-19, severe acute respiratory syndrome) as well as other inflammatory diseases (Crohn's disease, arthritis, psoriasis, atopic dermatitis).

The proposed invention may fill an existing gap in the therapeutic strategies for lower respiratory tract diseases, especially in patients who have already exhausted all remaining therapeutic options. At the same time, the described innovation is an alternative to a cell therapy relying on mesenchymal stem cells.

Using the preparation of MVs is an alternative to the utilization of whole cells that rely on an element of the secretome that reproduces their action, namely the secretory microvesicles (MVs). The use of this product enables us to omit the numerous problems resulting from the optimization of cell therapies. The yielded preparation of MVs is characterized by therapeutic potential in terms of limiting lower respiratory tract inflammation, and the positive effect of action thereof is retained over a longer period of time. The results of the preclinical studies thus convince that the preparation attained by the method acc. to the present invention may be an alternative in the treatment of asthma and other lower respiratory tract diseases.

### REFERENCES

Alashkar Alhamwe B, Potaczek D P, Miethe S, Alhamdan F, Hintz L, Magomedov A, Garn H. Extracellular Vesicles and Asthma-More Than Just a Co-Existence. Int J Mol Sci. 2021 May 7; 22 (9): 4984.
de Castro L L, Xisto D G, Kitoko J Z, Cruz F F, Olsen P C, Redondo P A G, Ferreira T P T, Weiss D J, Martins M A, Morales M M, Rocco P R M. Human adipose tissue mesenchymal stromal cells and their extracellular vesicles act differentially on lung mechanics and inflammation in experimental allergic asthma. Stem Cell Res Ther. 2017 Jun. 24; 8 (1):151.
Lee R H, Pulin A A, Seo M J, Kota D J, Ylostalo J, Larson B L, et al. Intravenous hMSCs improve myocardial infarction in mice because cells embolized in lung are activated to secrete the anti-inflammatory protein TSG-6. Cell Stem Cell 2009; 5:54-63
Müzes G, Sipos F. Mesenchymal Stem Cell-Derived Secretome: A Potential Therapeutic Option for Autoimmune and Immune-Mediated Inflammatory Diseases. Cells. 2022 Jul. 26; 11 (15):2300.
Shahir M, Mahmoud Hashemi S, Asadirad A, et al. Effect of mesenchymal stem cell-derived exosomes on the induction of mouse tolerogenic dendritic cells. J Cell Physiol. 2020; 235:7043-7055.
Wei, X., Yang, X., Han, Zp. et al. Mesenchymal stem cells: a new trend for cell therapy. Acta Pharmacol Sin 34, 747-754 (2013).
Wei et at. Mesenchymal stem cells: a new trend for cell therapy. Acta Pharmacologica Sinica (2013) 34:747-754

1. A method of producing a preparation of secretory microvesicles (MV) from mesenchymal stem cells (MSCs), characterized in that it comprises all the following steps:
a) a tissue previously isolated from the human body is subjected to, preferably mechanical, fragmentation,
b) the tissue fragmented in step a) is digested enzymatically, whereupon optionally the enzyme is inactivated,
c) large tissue fragments are separated, and then the remaining cell suspension is centrifuged, preferably at a speed of 300-1000×g at a time of 2-15 minutes, most preferably at 400×g for 5 minutes, at room temperature to obtain a pellet of MSC cells,
d) the MSC cells obtained in step c) are cultured in an MSC-dedicated culture medium supplemented with an antibiotic, wherein the cells are subjected to, at least triple, passaging at a temperature of 34-39 °C, preferably of 37 °C,
e) the MSC cells obtained as a result of step d) are cultured in the MSC-dedicated culture medium with the addition of at least one human cytokine selected from a group including IL-1β, TNFα, IFNγ, and mixtures thereof at a temperature of 34-39 °C, preferably at 37 °C,
f) the culture medium with the MSC cells from step e) is pre-purified by pre-centrifugation at 300×g-500×g for at least 3 minutes, preferably for 10 minutes, at room temperature, and then by subsequent centrifugation at a speed comprised in the range of 1000×g-3000×g for at least 3 minutes, preferably for 10 minutes, at room temperature, to obtain a supernatant of the culture medium, containing the secretory microvesicles,
g) optionally, the supernatant of the culture medium, containing the secretory microvesicles, from step f) is frozen at a temperature of −20 °C or lower for the purpose of storage, and after a desired amount of time, the culture medium is defrozen,
h) the supernatant of the culture medium, containing the secretory microvesicles, is subjected to at least one ultraviolet centrifugation to obtain the preparation of secretory microvesicles in the form of a precipitate.

2. The method according to claim 1, characterized in that in step a), the fragmentation is performed by cutting.

3. The method according to claim 1, characterized in that in step b), the enzyme is a collagenase, preferably collagenase I or collagenase IV or collagenase A or collagenase VII, or another enzyme with proteolytic activity, preferably a trypsin or a hyaluronidase or a serine protease.

4. The method according to claim 3, characterized in that in step b), collagenase IV, preferably at a concentration of 1 mg/mL, is used as the enzyme, wherein the fatty tissue with collagenase IV is subjected to incubation for at least 45 minutes at 37 °C, under an atmosphere of 5% CO₂ saturation with continuous mixing.

5. The method according to claim 4, characterized in that collagenase IV is inactivated by adding an inactivating agent in a ratio of 1:1 relative to the volume of collagenase IV, wherein the inactivating agent comprises a DMEM medium.

6. The method according to claim 1, characterized in that in step c) the large tissue fragments are separated by filtration, preferably using a filter with pores of from 40 μm to 100 μm.

7. The method according to claim 1, characterized in that in step d), the antibiotic is gentamicin.

8. The method according to claim 1, characterized in that in step d), IL-1β at a concentration of 1-100 ng/ml or TNFα at a concentration of 1-100 ng/ml or IFNγ at a concentration of 1-100 ng/ml or mixtures thereof are used as the human cytokine, wherein the culturing in step d) is conducted for at least 72 h, preferably for 96 h.

9. The method according to claim 8, characterized in that in step d), IL-1β at a concentration of 25 ng/ml or TNFα at a concentration of 50 ng/ml or IFNγ at a concentration of 50 ng/mL or mixtures thereof are used as the human cytokine.

10. The method according to claim 9, characterized in that in step d), a mixture comprising IL-1β at a concentration of 25 ng/ml, TNFα at a concentration of 50 ng/ml and IFNγ at a concentration of 50 ng/ml is used as the human cytokine.

11. The method according to claim 1, characterized in that in step f), the culture medium is subjected to pre-centrifugation at room temperature at a speed of 300×g for 10 minutes, and then to a subsequent centrifugation at room temperature at a speed of 2000×g for 10 minutes.

12. The method according to claim 1, characterized in that in step h), the culture medium is subjected to ultracentrifugation, preferably at 100,000×g for 60 minutes at 4 °C, to obtain the microvesicles in the form of a pellet, wherein the ultracentrifugation is repeated, and after the first ultracentrifugation, the pellet is rinsed, preferably with a concentrated buffered saline solution.

13. The method according to claim 1, characterized in that after step h), the precipitate of microvesicles is suspended in the buffered saline solution, whereupon optionally the suspension is frozen at −80 °C.

14. The method according to claim 1, characterized in that the tissue is an adipose tissue which is derived from fatty skin folds taken from a patient after an abdominoplasty procedure, wherein the patient is preferably a bariatric patient.

15. The method according to claim 1, characterized in that the tissue is derived from Wharton's jelly.

16. The preparation of secretory microvesicles of mesenchymal stem cells, as produced by the method of claims 1 to 15, for use as a drug.

17. The preparation of secretory microvesicles of mesenchymal stem cells, according to claim 16, for use in the treatment of inflammatory lower respiratory tract diseases, preferably chronic obstructive pulmonary disease or COVID-19 or idiopathic pulmonary fibrosis or severe asthma.
